(19) United States
(12) Patent Application Publication
(10) Pub. No.: US 2015/0051191 A1
(43) Pub. Date: Feb. 19, 2015

(54) TREATMENT OF ALCOHOLISM USING IBUDILAST

(71) Applicant: MEDICINOVA, INC., La Jolla, CA (US)

(72) Inventors: Yuichi IWAKI, La Jolla, CA (US); Kirk JOHNSON, Moraga, CA (US)

(73) Assignee: MEDICINOVA, INC., La Jolla, CA (US)

(21) Appl. No.: 14/460,207

(22) Filed: Aug. 14, 2014

Related U.S. Application Data
(60) Provisional application No. 61/866,205, filed on Aug. 15, 2013.

(51) Int. Cl. A61K 31/437; A61K 45/06

(52) U.S. Cl. CPC A61K 31/437; A61K 45/06
USPC 514/211.13; 514/300; 514/282; 514/250

(57) ABSTRACT

Alcoholism, and symptoms and negative effects thereof may be treated using ibudilast or a pharmaceutically acceptable salt thereof. Abstinence from alcohol consumption may be maintained using ibudilast or a pharmaceutically acceptable salt thereof. Withdrawal from alcohol may be facilitated and negative effects thereof reduced by using ibudilast or a pharmaceutically acceptable salt thereof.

### CROSS-REFERENCE TO RELATED APPLICATION

This application claims priority under 35 U.S.C. section 119(e) to U.S. Provisional Application No. 61/866,205 filed Aug. 15, 2013, which is incorporated herein by reference in its entirety.

### BACKGROUND OF THE INVENTION

Excessive consumption of alcohol is a major health concern globally. Alcoholism is considered to be a chronic disease, a drug addiction, a learned response to crisis, a symptom of an underlying psychological or physical disorder, or a combination of these factors and is marked by repeated alcohol use despite a host of negative, physical and psychosocial, effects.

Most approaches to the treatment of alcoholism require the alcoholic person to recognize his/her illness and to abstain from alcohol. Treatment programs can include combinations of: psychological rehabilitative treatments, organized self-help groups, aversion therapy based on behavior modification, injections of vitamins or hormones, and the use of abstinence-maintaining drugs.

To date, only a few agents are approved by the Food and Drug Administration (FDA) for the treatment of alcoholism and these agents are only modestly effective. While drugs such as acamprosate (CAMPRAL), ondansetron (ZOFRAN), naltrexone (VIVITROL), disulfiram (ANTABUSE) and calcium citrate cyanamide are used as therapeutics for treating alcoholism, these therapeutic agents have drawbacks related to their efficacy and associated side effects. Provided herein are methods and compositions related to the use of ibudilast to treat alcoholism, to maintain abstinence from alcohol, or to reduce alcohol consumption at a level that is not harmful or less harmful to a person's health.

### SUMMARY OF THE INVENTION

The present technology is directed to methods of managing alcohol-dependent or alcohol-abusing patients who want to or need to remain in a state of enforced sobriety so that supportive and psychotherapeutic treatment may be provided to best assist treatment. The present technology is also directed to methods that will affect the drinking pattern of a chronic alcoholic, preferably, lowering if not eliminating the number of alcoholic drinks consumed by the subject compared to pre-treatment levels or control subjects. In other embodiments the present technology is also directed to a treatment of alcohol dependence. Stated another way, the present technology is directed to methods for the engagement and maintenance of abstinence from alcohol consumption in patients with alcohol dependence.

In one embodiment, a method is provided for treating alcoholism and/or alcohol dependence in a subject diagnosed with alcoholism and/or alcohol dependence, the method including administering to the subject a therapeutically effective amount of ibudilast. As used herein, and unless the context indicates otherwise, ibudilast includes a pharmaceutically acceptable salt of ibudilast. In another embodiment, provided herein is a method of treating alcoholism or a symptom thereof in a subject diagnosed with alcoholism, the method including administering to the subject a therapeutically effective amount of ibudilast. In another embodiment, a method is provided for maintaining abstinence from alcohol consumption in a subject in need of, the method including administering to the subject a therapeutically effective amount of ibudilast. In another embodiment, a method is provided for treating a subject suffering from withdrawal from alcohol or a symptom thereof, the method including administering to the subject a therapeutically effective amount of ibudilast. In another embodiment, a method is provided for reducing alcohol consumption in a patient suffering from alcoholism, the method including administering to the subject a therapeutically effective amount of ibudilast.

According to another embodiment, a method is provided for facilitating maintenance of abstinence from alcohol consumption in a subject having such a need, the method including administering to the subject a therapeutically effective amount of ibudilast. Pursuant to the method, the subject undergoing treatment has been alcohol-free for 10 days or more, 20 days or more, or 30 days or more prior to receiving ibudilast and continues to remain alcohol free for at least another 30 days, at least another 60 days, or at least another 120 days upon receiving ibudilast. According to the method, ibudilast can be administered once, twice, thrice, or four times daily, without substantially altering the subject's daily intake of water.

In one embodiment, ibudilast is formulated as a delayed-release tablet or capsule containing from 10 mg to 50 mg of ibudilast. The delayed-release formulation is such that the plasma half-life of ibudilast is about 19 hours.

In another embodiment, a method is provided for treating a subject suffering from withdrawal from alcohol, the method including administering to the subject a therapeutically effective amount of ibudilast. In certain embodiments, treatment with ibudilast reduces or eliminates behavioral changes selected from the group consisting of feelings of nervousness, hyperexcitability, sleep disturbances and dysphoric mood for a time period of at least 5 days, to at least 180 days.

In some embodiments, ibudilast can be administered orally as a therapeutically effective daily dose from about 3 mg to about 120 mg, for instance a therapeutically effective daily dose of 100 mg. As used herein, the amounts refers to amounts of ibudilast or the free base equivalent amount thereof of a pharmaceutically acceptable salt of ibudilast. The therapeutically effective daily dose may be administered as a single dose or it can be divided in two doses of 50 mg each. Alternatively, the therapeutically effective daily dose may be divided into three or four doses.

For certain embodiments, the therapeutically effective daily dose is 80 mg or less and is divided into two doses. The method also encompasses a combination therapy where one or more of other therapeutic agents may be administered along with ibudilast. Non-limiting examples of other therapeutic agents suitable for the treatment of alcoholism and/or alcohol dependence are those selected from acamprosate, naltrexone, quetiapine, disulfiram, ondansetron, or levatiracetam.

### BRIEF DESCRIPTION OF THE DRAWINGS

FIG. 1 graphically illustrates the mean ethanol intakes for P (upper panels) and HAD1 (lower panels) rats during the maintenance (left side of panel) and relapse (right side of panel) test-phases. * indicates the respective dose differed significantly from vehicle (p<0.05). Grey bar indicates the vehicle value.

FIG. 2 graphically illustrates the mean ethanol intakes for Chronic Intermittent Ethanol (EtOH) exposed mice and control (CTL) mice during Test Cycles 7, 8, 9. For comparison purposes, the horizonal dashed line indicates mean ethanol intake by vehicle-treated CTL mice during test cycle 7. * indicates less ethanol intake compared to vehicle-treated mice (p<0.05). # indicates greater intake than CTL group (p<0.05).

FIG. 3 is a flow chart illustrating human Phase I clinical trial protocol.

### DETAILED DESCRIPTION

The practice of the present technology will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed. All publications, patents and patent applications cited herein, are hereby incorporated by reference in their entirety.

As used herein, and in the appended claims, the singular forms “a,” “an” and “the” include plural references unless the context clearly dictates otherwise.

“Administering” or “administration of” a drug to a patient (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

“Comprising” shall mean that the methods and compositions include the recited elements, but not exclude others. “Consisting essentially of” when used to define methods and compositions, shall mean excluding other elements of any essential significance to the combination for the stated purpose. “Consisting of” shall mean excluding more than trace elements of other ingredients and substantial method steps for administering the compositions utilized or provided herein or process steps to produce a composition or achieve an intended result. Embodiments defined by each of these transitional terms and phrases are within the scope of this technology.

“Ibudilast” refers to a compound of formula:

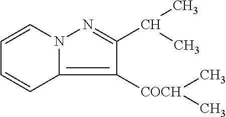

“Pharmaceutically acceptable” refers to safe and non-toxic for the methods and compositions provided herein, e.g., for in vitro or in vivo administration, preferably to mammals.

A “pharmaceutically acceptable salt” is a pharmaceutically acceptable, organic or inorganic acid or base salt of a compound. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the lowering or reduction of, or amelioration or eradication of a disease or one or more symptoms associated with a disease. In certain embodiments, such terms refer to minimizing the spread or worsening of the disease resulting from the administration of one or more prophylactic or therapeutic agents to a patient with such a disease. For purposes of the various aspects and embodiments provided herein, “treatment” includes, but are not limited to, reduction, alleviation, or amelioration of one or more manifestations of or negative effects of the disease or condition treated, improvement in one or more clinical outcomes, diminishment of extent of disease, delay or slowing of disease progression, amelioration, palliation, or stabilization of the disease state, and other beneficial results described herein.

The term “effective amount” refers to an amount of a compound sufficient to provide a therapeutic or prophylactic benefit in the treatment or prevention of a disease or to delay or minimize symptoms associated with a disease. Further, a therapeutically effective amount with respect to a compound utilized herein means that amount of the compound alone, or in combination with other therapies, that provides a therapeutic benefit in the treatment or prevention of a disease. Used in connection with a compound utilized herein, the term can encompass an amount that improves overall therapy, reduces or avoids symptoms or causes of disease, or enhances the therapeutic efficacy of or synergies with another therapeutic agent. The full therapeutic effect does not necessarily occur by administration of one dose (or dosage), and may occur only after administration of a series of doses. Thus, an effective amount may be administered in one or more administrations.

A “subject” or “patient” may include an animal, such as a human, cow, horse, sheep, lamb, pig, chicken, turkey, quail, cat, dog, mouse, rat, rabbit or guinea pig. The animal can be a mammal such as a non-primate and a primate (e.g., monkey or human). In one embodiment, the patient is a human.

“Alcoholism” or “alcohol dependence” is a chronic and often progressive disease that includes problems controlling one's drinking, being preoccupied with alcohol, continuing to use alcohol even when it causes problems, having to drink more to get the same effect (physical dependence), or having withdrawal symptoms when you rapidly decrease or stop drinking Alcoholism is considered to be a chronic disease, a drug addiction, a learned response to crisis, a symptom of an underlying psychological or physical disorder, or a combination of these factors and is marked by repeated alcohol use despite a host of negative physical and psychosocial effects. Signs and symptoms of alcoholism include, without limitation: to be unable to limit the amount of alcohol one drinks; feel a strong need or compulsion to drink; develop tolerance to alcohol so that you need more to feel its effects; drink alone or hide one' drinking; experience physical withdrawal symptoms—such as nausea, sweating and shaking—when one does not drink; forget conversations or commitments, sometimes referred to as a “black out;” make a ritual of having drinks at certain times and become annoyed when this ritual is disturbed or questioned; be irritable when your usual drinking time nears, especially if alcohol is not available; keep alcohol in unlikely places at home, at work or in one's car; gulp drinks, order doubles or become drunk intentionally to feel good, or drink to feel “normal;” have legal problems or problems with relationships, employment or finances due to drinking; and lose interest in activities and hobbies that used to bring you pleasure.

“Alcohol withdrawal” syndrome or “withdrawal from alcohol” is a potentially life-threatening condition that can occur in people who have been drinking heavily for weeks, months, or years and then either stop or significantly reduce their alcohol consumption. Alcohol withdrawal symptoms can begin as early as two hours after the last drink, persist for weeks. Symptoms range from mild anxiety and shakiness to severe complications, such as seizures and delirium tremens (also called DTs). The death rate from DTs—which are characterized by confusion, rapid heartbeat, and fever—is estimated to range from 1% to 5%. Appropriate alcohol withdrawal treatments can reduce the risk of developing withdrawal seizures or DTs. Severe alcohol withdrawal symptoms are a medical emergency.

“Abstinence from alcohol consumption” refers to complete abstinence or a reduction in alcohol consumption in a patient in need of abstaining from alcohol or reducing the patient's alcohol consumption.

The results described below demonstrate ibudilast's ability to decrease voluntary ethanol consumption, under blind testing conditions, in selectively bred alcohol-preferring (P) and high-alcohol-drinking (HAD1) rats. The ability of ibudilast to treat alcohol dependence using a mouse model of ethanol dependence was tested; the which model involved repeated cycles of chronic intermittent ethanol (CIE) exposure (see Litten et al. Addict. Biol., 17:513-527, (2012). Because elevated intake of alcohol is characteristic of animals used in each of the above mentioned models and such an increase in alcohol intake is contemplated to result from biological mechanisms relevant to human alcohol dependence, the above-mentioned models are contemplated to be suitable for testing the utility of ibudilast for reducing alcohol intake in humans. See Egli et al., Addict. Biol., 10:309-319, (2005)

Results using alcohol-preferring and high-alcohol-drinking rats indicate that ibudilast reduces ethanol intake in rats in both groups by approximately 50% as compared to control rats. As further explained below in the Examples section, the test was designed to measure the ability of ibudilast to decrease voluntary ethanol consumption in four phases: an initial maintenance test phase, an alcohol drinking recovery phase, an alcohol deprivation phase and an alcohol reintroduction (relapse) phase.

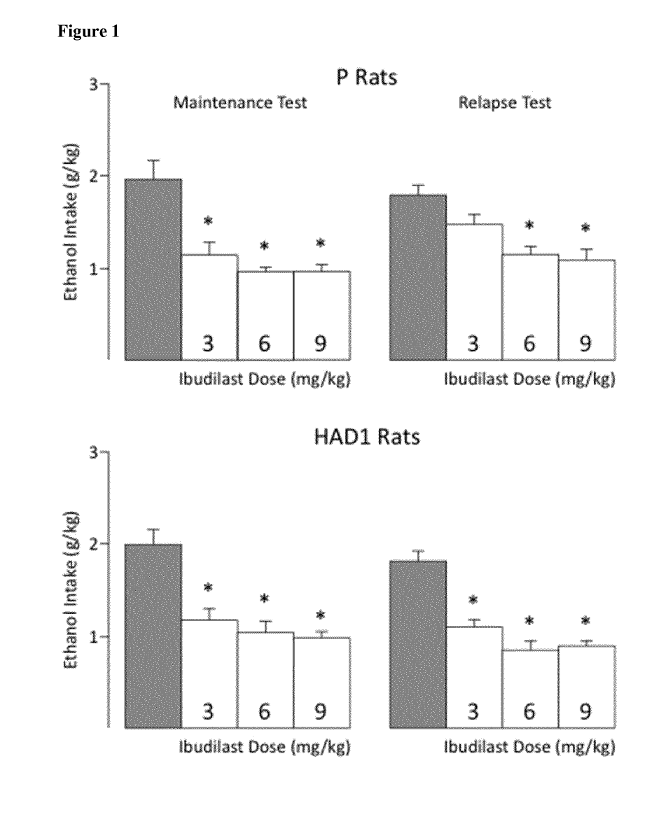

As illustrated in FIG. 1, ibudilast decreased ethanol intake in a dose dependent manner in both alcohol-preferring and high-alcohol-drinking rats during the maintenance and relapse phases. Ethanol intake levels were elevated, however, during the recovery phase when no treatment is administered.

A correlation between the dose of ibudilast and number of days over which treatment was administered is also observed for alcohol-preferring and high-alcohol-drinking rats. The ability of ibudilast to reduced intake of ethanol was strongest on the first day of the relapse phase and ethanol intake levels increased gradually in both groups of rats on days 2-5 of the relapse test phase.

Ibudilast also reduced ethanol intake in adult male C57BL/6J mice model of alcohol dependence. Briefly, mice were divided into test (ethanol dependent) and control (non-ethanol dependent) groups. Mice in the former group were exposed to chronic intermittent ethanol (CIE) vapor (16 hr/day×4 days), and then forced to abstain from alcohol for 72 hours. Mice were then permitted access to ethanol for 2 h/day for a 5-day test period. This pattern of weekly CIE exposures followed by 5-day test periods was repeated for 9 cycles. Mice in the control group were treated in the same manner, except these animals were exposed to air rather than ethanol vapors. Mice from both groups were further divided into four sub-groups with animals receiving 0 mg/Kg, 3 mg/Kg, 6 mg/Kg or 12 mg/Kg dose of ibudilast.

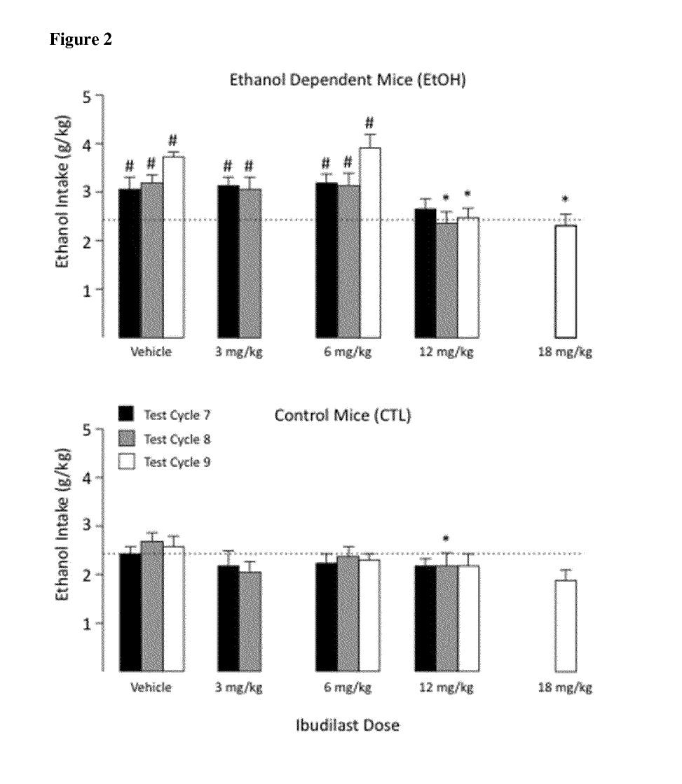

As illustrated in FIG. 2, ibudilast reduced ethanol intake in both control and test groups. The therapeutic effect of ibudilast was stronger in the test group and the therapeutic efficacy increased over repeated testing and drug treatment cycles starting from cycle 7. Surprisingly, at the higher doses, ibudilast decreased ethanol intake of mice in the test group to ethanol intake levels below those for mice in the control group.

Human studies are also contemplated to confirm that ibudilast is useful for the treatment of alcoholism and alcohol dependence in human subjects requiring such therapy. The human studies can test that (1) ibudilast at a dose of 50 mg twice a day (BID) does not adversely alter the cardiovascular response in human patients who are administered alcohol intravenously; (2) ibudilast at a dose of 50 mg twice a day alters subjective response to intravenously administered alcohol and alcohol-induced cravings; and (3) ibudilast at a dose of 50 mg twice a day alters stress-induced and cue-induced cravings for alcohol. The study parameters, criteria used to enroll patients and the end points of the human studies are further explained in the Examples section below. A useful dose range of ibudilast is 10-500 mg—administered once a day, twice a day, or three times a day. Specific dose amounts are also contemplated, which are 10, 15, 25, 30, 50, 75, 100, 150, 200, 250, 300, 400 and 500 mg.

In addition to ibudilast, the treatment of alcoholism and alcohol dependence may optionally include other drugs. Exemplary of such drugs without limitation are acamprosate, naltrexone, quetiapine, disulfiram, ondansetron, varenicline, topiramate, prazocin, sertraline and levatiracetam. These agents can be administered together with ibudilast or separately.

In one aspect, a method is provided for maintaining abstinence from alcohol in a subject with alcohol dependence by administering to such a subject a therapeutically effective amount of ibudilast. In one embodiment, “maintaining abstinence from alcohol” refers to the ability of a therapeutic agent to prevent the subject undergoing treatment from consuming alcohol.

According to another embodiment, a therapeutically effective dose of ibudilast alone or in combination with one or more additional drugs reduces the frequency of alcohol consumption, reduces the number of alcoholic drinks consumed per day and/or reduces the number of days alcohol is consumed in a month. For instance, treatment with ibudilast may reduce the number of alcoholic drinks consumed per day from greater than 5 drinks/day to less than 4 drinks/day, less than 3 drinks/day, less than 2 drinks/day, less than 1 drink/day, or completely eliminate the urge to consume alcohol.

The ability of ibudilast to maintain abstinence from alcohol may also be evaluated by measuring the number of days a subject refrains from consuming alcohol. According to one embodiment, ibudilast prevents the consumption of alcohol for at least 30 days, at least 60 days, at least 90 days, at least 120 days, at least 150 days, at least 180 days, at least 210 days, at least 240 days, at least 270 days, at least 300 days, at least 330 days, or greater than 365 days.

In another aspect, a method is provided for attenuating or eliminating the effects of withdrawal from alcohol or treating a subject suffering from withdrawal from alcohol by administering a therapeutically effective dose of ibudilast. Withdrawal from alcohol is a condition marked by feelings of nervousness, hyperexcitability, sleep disturbances and dysphoric mood.

Therapeutic efficacy can be judged by measuring a lowering in the number of incidences of nervousness a subject experiences in a specific study period. Additionally, reduction or elimination of episodes of hyperexcitability, a reduction or elimination sleep disturbances and dysphoric mood swings in a subject receiving treatment can be used as metrics for judging therapeutic efficacy of ibudilast.

In one embodiment, ibudilast will reduce or eliminate behavioral changes accompanying withdrawal from alcohol for at least 5 days, at least 10 days, at least 20 days, at least 30 days, at least 60 days, at least 90 days, at least 120 days, at least 180 days, or for a time period exceeding 180 days. As described above, ibudilast may be administered as a single daily dose or the daily dose may be divided in two, three or more separate doses that can be administered at specific intervals of time depending on the plasma concentration levels needed for therapeutic efficacy. The dose of ibudilast can be modified depending on the age, physical and mental condition of the patient being treated, the severity of the withdrawal effects as assessed by the prescribing physician.

In another aspect, a pharmaceutical composition is provided, the composition including a therapeutically effective amount of ibudilast or a pharmaceutically acceptable prodrug, or salt thereof and a pharmaceutically acceptable carrier for treating alcoholism and/or alcohol dependence.

Pharmaceutical compositions provided herein encompass, in some embodiments, formulations suitable for systemic administration. Oral dosage forms include tablets, lozenges, capsules, syrups, oral suspensions, emulsions, granules, and pellets. Alternative formulations include transdermal patches, powders or lyophilates that can be reconstituted with the help of a suitable diluent. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof.

Formulations suitable for parenteral administration include aqueous and non-aqueous isotonic sterile solutions suitable for injection, as well as aqueous and non-aqueous sterile suspensions. Parenteral formulations provided herein are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use.

A formulation provided herein is a sustained release formulation suitable for oral or perenteral delivery. The sustained release compositions can contain ibudilast alone or a combination of ibudilast and a second drug. For such compositions, each drug is released or absorbed slowly over time, when compared to a non-sustained release formulation. Sustained release formulations may employ pro-drug forms of the active agent, delayed-release drug delivery systems such as coatings, liposomes, polymer matrices, or hydrogels.

The pharmaceutically acceptable carrier material can be an organic or inorganic inert carrier material, for example one that is suitable for oral administration. Suitable carriers include water, gelatin, gum arabic, lactose, starch, magnesium stearate, talc, vegetable oils, polyalkylene-glycols, petroleum jelly and the like that are used to manufacture tablets, capsules, lozenges and the like. Furthermore, the pharmaceutical preparations may also contain other pharmaceutical additives such as flavoring agents, preservatives, stabilizers, emulsifying agents and buffers. These agents are added in accordance with accepted practices of pharmaceutical compounding.

The compositions provided herein deliver a therapeutically effective dose of ibudilast. In this context, a therapeutically effective dose of ibudilast will depend on the severity of the condition being treated, the medical history of the patient being treated, the age and weight of the person undergoing treatment and will typically range from a dose of about 3 mg/day to about 300 mg/day.

The dosing regimen can be altered by the attending physician depending on the patients needs. Treatment methods provided herein encompass the administration of ibudilast as a single dose, as two daily doses, as three daily doses, as four daily doses, as five daily doses for a time course of one day to several days, weeks, months, and even years. Exemplary dosing schedules include, without limitation, administration five times a day, four times a day, three times a day, twice daily, once daily, every other day, three times weekly, twice weekly, once weekly, twice monthly, once monthly, and so forth.

In some embodiments, ibudilast is administered daily as a single therapeutically effective dose. Exemplary daily doses include without limitation, a dose of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg.

For certain treatment regimens, ibudilast is administered as two daily doses with each dose containing about 3-150 mg of ibudilast. For instance, each dose can contain about 5 mg of ibudilast, about 10 mg of ibudilast, about 20 mg of ibudilast, about 30 mg of ibudilast, about 40 mg of ibudilast, about 50 mg of ibudilast, about 60 mg of ibudilast, about 70 mg of ibudilast, about 80 mg of ibudilast, about 90 mg of ibudilast, about 100 mg of ibudilast, about 110 mg of ibudilast, about 120 mg of ibudilast, about 130 mg of ibudilast, about 140 mg of ibudilast, or about 150 mg of ibudilast.

### EXAMPLES

### Example 1

### Evaluation of Ibudilast as a Therapeutic Agent for Decreasing Voluntary Alcohol Consumption in Mice

### Example 1-A

The ability of ibudilast (MN-166) to decrease voluntary ethanol consumption, under blind testing conditions, in selectively-bred alcohol-preferring (P) and high-alcohol-drinking (HAD1) rats and in a mouse model of ethanol dependence was examined. While each model is characterized by elevated alcohol intake, drugs such as quetiapine and levatiracetam do not selectively reduce ethanol drinking in these models.

To perform the study, adult male P and HAD1 rats were randomly assigned to receive one of the following four doses of ibudilast—0 mg/Kg, 3 mg/Kg, 6 mg/Kg, or 9 mg/Kg with eight rats assigned to each dose. Each dosing group was balanced to average a 2 h/day ethanol (15% v/v) intake. Water was concurrently available to the rats and Mazola corn oil was used as the vehicle.

The study was divided into four test phases. In the maintenance test phase rats in the treatment group were injected ibudilast subcutaneously (2 mL/kg s.c.), using standard solutions that deliver 3 mg/Kg, 6 mg/Kg or 9 mg/Kg of ibudilast, 60 min before each ethanol test session and the same dose of ibudilast was administered subcutaneously to the rats 8 hours later. Rats in the control group, however, are administered 2 mL/Kg Mazola corn oil subcutaneously. This protocol was carried out for 4 consecutive days. Following the maintenance test phase, is a two week no drug recovery phase during which rats are permitted access to ethanol and the amount of ethanol consumed by the rats was measured.

The recovery phase is followed by a two week forced ethanol abstinence phase. Following forced abstinence, the effects of ibudilast on ethanol drinking were examined by re-introducing ethanol to rats for a period of 5 consecutive days (i.e., the Relapse Test phase). Each animal received the same dose of ibudilast during maintenance and relapse test phases.

FIG. 1 illustrates the results of this study. Thus, it is observed that ibudilast reduced ethanol intake by approximately 50% in both P and HAD1 rats (FIG. 1, left panel), during the maintenance test phase. Separate 2-way mixed ANOVA analysis indicates a dose dependent reduction of ethanol intake for P [F(3.28)=12.425, p<0.001] and HAD1 [F(3.28)=14.943, p<0.001] rats. Each of the four doses of ibudilast reduced ethanol intake over the 4-day maintenance test-phase relative to vehicle-injected controls (p's≦0.001).

Ibudilast also reduced ethanol intake in P and HAD1 rats by about 50% during the 5-day Relapse Test phase (FIG. 1, right panel). Separate 2-way mixed ANOVA analysis indicates a dose dependent reduction in ethanol intake for P rats [F(3.28)=8.483, p<0.001], with the 6 mg/Kg and the 9 mg/Kg doses significantly reducing ethanol drinking relative to controls (p<0.05), and HAD1 rats [F(3.28)=25.801, p<0.001]. Indeed, each test dose of ibudilast was found to reduce ethanol intake compared to controls (p<0.05).

Significant Dose×Day interactions in P [F(12.112)=3.257, p<0.001] and HAD1 [F(12.112)=2.094, p=0.023] lines indicated that reduced ethanol intake by ibudilast was most robust on the first day when the drug was administered (data not shown).

### Example 1-B

In a separate study, adult male C57BL/6J mice were used to evaluate ibudilast as a therapeutic for the treatment of alcohol dependence. Two groups of mice were used for the study. Mice in the first group were trained to drink ethanol using a 2 h/day free-choice (15% v/v ethanol) drinking procedure (ethanol dependent (EtOH) mice), while mice in the second group were provided water and served a control (ethanol nondependent (CTL)) mice. Both the ethanol dependent and ethanol non-dependent groups had equal number of mice (N=37-38/group).

Mice in the alcohol dependent group were then exposed to chronic intermittent ethanol (CIE) vapors for 16 hr/day over four days. After a 72 hours forced abstinence period, the ethanol dependent mice were again permitted access to ethanol for 2 hours each day over a 5 consecutive test days. This pattern of chronic intermittent ethanol vapor exposure for four consecutive days followed by forced abstinence and a five day ethanol access test period was repeated for 9 cycles. Mice in the control (CTL) group were treated in a similar manner except that air was used in the inhalation chambers in place of ethanol vapors.

Both control and ethanol dependent mice received subcutaneous (s.c.) injections of the vehicle at 9 hours and 1 hour prior to the start of daily drinking sessions during Test Cycles 4, 5 and 6 to acclimate the animals to handling and administration of injections.

At the end of test cycle 6, mice in the ethanol dependent group and the control group were separated into four sub-groups with animals receiving one of four doses of ibudilast—0 mg/Kg, 3 mg/Kg, 6 mg/Kg or 12 mg/Kg during Test Cycles 7 and 8. Each sub-group contained 9-10 mice (N=9-10/group). The dose of ibudilast was increased during Test Cycle 9 with ethanol dependent and control mice receiving one of the following four doses of ibudilast—0 mg/Kg, 6 mg/Kg, 12 mg/Kg, or 18 mg/Kg. Thus, mice previously receiving a dose of 3 mg were administered ibudilast at a dose of 18 mg/Kg during Test Cycle 9.

It was observed that ethanol intake by mice increased with successive exposures to chronic intermittent ethanol vapors. For control mice, however, ethanol intake remained relatively the same throughout the nine Test Cycles of the study. Thus, alcohol dependent mice consumed significantly greater quantities of ethanol than control mice starting at Test Cycle 6 (main effect of Group [F(1.67)=35.84, p<0.001), and this effect persisted during Test Cycle 7 [F(1.67)=21.80, p<0.001], Test Cycle 8 [F(1.67)=12.52, p<0.001], and Test Cycle 9 [F(1.66)=32.55, p<0.001].

Interestingly, the therapeutic efficacy of ibudilast to lower ethanol consumption appeared to increase with repeated cycles of exposure to chronic intermittent ethanol vapors and drug treatment. As illustrated in FIG. 2, ibudilast at a dose of 12 mg/Kg reduced ethanol in alcohol dependent mice. The same dose of ibudilast reduced ethanol consumption to a greater extent during Test Cycle 8, [F(3.67)=3.36, p<0.05] in both alcohol dependent and control mice relative to mice receiving vehicle (0 mg of ibudilast) in the alcohol dependent group and the control group respectively (p<0.05).

The data in FIG. 2 further illustrates that during Test Cycle 9 ibudilast at a dose of 12 mg/Kg and 18 mg/Kg was more effective at reducing ethanol intake in alcohol dependent mice than control mice (Group×Dose interaction: [F(3.66)=4.50, p<0.01]). Pair-wise comparisons indicated that alcohol dependent mice treated with vehicle or 6 mg ibudilast consumed significantly more ethanol than their respective counterparts in the control group (p<0.05). In contrast, there was no significant difference in ethanol intake for alcohol dependent mice and control mice receiving ibudilast at a dose of 12 mg/Kg and 18 mg/Kg. These results indicate that ibudilast at a dose of 12 mg/Kg or 18 mg/Kg is effective at reducing ethanol intake in alcohol dependent mice to ethanol intake levels for mice in the control group (FIG. 2).

Decreased ethanol intake, moreover, does not result from a general suppression of ingestive behavior. Ibudilast was observed to reduce ethanol drinking in alcohol dependent mice at doses that did not affect ethanol drinking in mice in the control group. Moreover, decreased ethanol intake was associated with significant increases in concurrent water intake. While Ibudilast, especially at the 9 mg/Kg dose, produced transitory reductions in 24-hour food intake, but not water intake in P and HAD rats this effect diminished over the 5-day test phase.

### Example 2

### Evaluation of the Safety, Tolerance and Efficacy of Ibudilast in Human Alcoholics

Human studies are contemplated to test that: (1) ibudilast (50 mg BID) does not adversely alter the cardiovascular response to alcohol administered intravenously; (2) ibudilast (50 mg BID) alters the subjective response to alcohol administered intravenously and alcohol-induced cravings; and (3) ibudilast (50 mg BID) alters stress-induced and cue-induced craving.

To address each of the above mentioned specific aims, a randomized, double-blind, placebo-controlled study with subject crossover is to be conducted to determine the safety, tolerability, and efficacy of ibudilast. To be included in the study, participants will need to fulfill the following requirements—be between the ages of 21 and 65 years; meet the DSM-IV diagnostic criteria for alcohol dependence; have reported drinking at least 48 standard drinks in a 30-day period, during the 90 days before enrollment; and are non-treatment seeking alcohol dependent individuals that will consent to an inpatient stay at a medical facility during the course of the study.

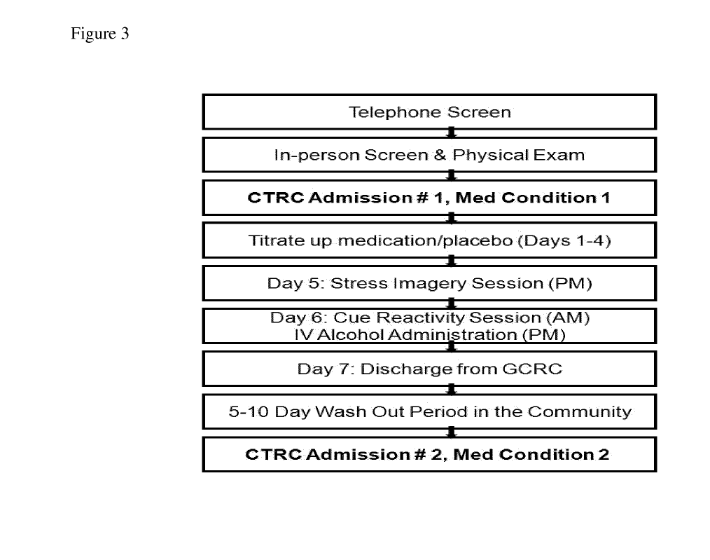

Initially, each subject will answer questions about quantity and frequency of drinking. Preliminary screening is by telephone using the Time Line Follow Back (TLFB) protocol. During the screening calls, subjects will be asked to provide smoking and drug use data. The present inventors have developed a medical history interview questionnaire to screen for medical conditions that contraindicate study participation. Following review of each participant's history along with laboratory tests by the study physician, participants will undergo a physical examination where vital signs, weight, and other parameters will be measured. Blood and urine samples will be collected to test: (a) LFTs, (b) glucose, (c) drugs, and (d) a blood chemistry screen will be performed. Female participants will consent to a urine pregnancy screen that will be performed prior to each intravenous alcohol administration. The flow chart in FIG. 3 illustrates the study protocol.

Briefly, alcohol dependent individuals will be treated with ibudilast or placebo (PBO). Ibudilast will be obtained in the form of capsules, e.g., and without limitation, containing a delayed-release 10 mg formulation. Matching capsules of the placebo also will be obtained. Each participant will complete two separate 7-day (6 nights) inpatient stays at a Clinical and Translational Research Center (CTRC) during which they will be administered ibudilast twice daily under direct observation of the study nurse. The study physician will conduct daily rounds and examinations of participants for the entire duration of the study. Side effects will be collected in an open-ended fashion and the Systematic Assessment for Treatment Emergent Events (SAFTEE) will be administered daily.

The study will comprise a treatment group and a placebo group and each participant will complete a daily assessment questionnaire to address changes in mood, withdrawal effects, alcohol cravings, and address issues pertaining to other side effects. Thus, non-treatment seeking alcohol dependent individuals will be treated with ibudilast 50 mg (5×10 mg capsules) twice daily (BID) or placebo (PBO). To minimize nausea, however, each participant in the test group will receive 20 mg of ibudilast twice a day (bid) on days 1 and 2 of the study. The dose will be increased to two separate doses of 50 mg each on days 3-6. Upon reaching a stable target dose of ibudilast (or placebo), participants will complete a stress-exposure paradigm on day 5 (PM), an alcohol cue-exposure on day 6 (AM), and an intravenous alcohol challenge on day 6 (PM). Following the intravenous alcohol challenge, participants will be monitored for 24 hours and then discharged. Following a 5-10 day washout period study participants will be readmitted to the CTRC, for the second part of the study. See FIG. 3.

a. Intravenous Alcohol Administration (IV-A)

Infusion will be performed by the study nurse under the supervision of the study physician using a 5% alcohol IV solution. An alcohol infusion nomogram provides a formula for obtaining the rate of infusion based on the participant's gender and weight. Thus, male participants' will be infused at a rate determined by the following formula: 0.166-ml/minute X weight in kilogram while the rate of infusion for female participants is calculated as—0.126-ml/minute X weight in kilograms.

Breath alcohol content (BrAC) will be monitored every 3 to 5 minutes during infusion until target BrAC's of 0.02 g·dl, 0.04 g·dl, 0.06 g·dl, and 0.08 g·dl are obtained, following which infusion rates will be reduced to half the original rate to maintain stable BrAC levels during the testing phase. The following parameters will be assessed prior to intravenous alcohol challenge and again at each target BrAC: (1) Subjective High Assessment Scale (SHAS) commonly used in alcohol challenge studies; (2) Biphasic Alcohol Effects Scale (BAES) to obtain a reliable and valid measure of alcohol's stimulant and sedative effects; (3) Alcohol Urge Questionnaire (AUQ)—an 8-item scale where subjects rate their craving for alcohol at the present moment, which is appropriate for examining the level of the urge to drink alcohol; and (4) Heart Rate and Blood Pressure levels measure at baseline and at each target level of BrAC.

b. Cue Reactivity Assessment (CR)

The present study also will assess the reactivity of participants to alcoholic cues at each dose of medication such that participants will serve as their own controls. Repeated cue assessments may be useful because studies have indicated that not all alcohol dependent patients are cue reactive. The CR assessments will follow well-established procedures and participants will receive standardized instructions about the assessment as they becoming acclimated to the psychophysiological monitors.

CR assessment sessions will begin with a 3-minute relaxation period, in which participants will be asked to sit quietly and do nothing. Participants will then hold and smell a glass of water for 3 minutes as a standard procedure to control for the effects of simple exposure to any potable liquid, followed by a second 3-minute relaxation period. Next, each participant will hold and smell a glass of their preferred alcoholic beverage for three 3-minutes. During each trial, the participant will be asked to sniff the beverage for 5 consecutive seconds upon hearing a tone sound. 13 tone sounds will be administered during each 3-minute block of time. The intervals between tone sounds will be varied to ensure that each participant receives the same olfactory exposure. The order in which participants sniff water or an alcoholic beverage of choice is not counterbalanced because of carryover effects that are known to occur and that would interfere with determination of CR. The water trial will provide a baseline that controls for all aspects of stimuli and movement except the nature of the beverage. Participants will be allowed a smoke break immediately prior to and immediately after the cue reactivity assessment.

Following each 3 minute exposure to alcohol, each participant will rate his/her urge to drink alcohol using the 11-point Likert scale (e.g., “none at all” to “extremely strong urge”). Heart rate (beats per minute; BPM) and blood pressure (systolic, diastolic, and mean arterial pressure [MAP]) will be monitored continuously using a Dynamap Adult Vital Signs Monitor and values will be averaged over each 3-minute trial. Therapeutic efficacy of ibudilast will be gauged based on the compound's ability to attenuate or abolish cue induced cravings for alcohol consumption.

c. Stress Reactivity Assessment (SR)

Personal information collected at the time of admission (day 1) to the CRTC will be used to generate personalized scripts of neutral and stressful conditions. Participants will be asked to identify a recent stressful experience and to rate them on a 0 to 10 Likert scale, where a rating of 10 is considered to be the most stressful experience. Only stressful events rated ≧8 will be used in script development. Data on physical symptoms associated with the stressful and neutral events will also be collected for script development.

During the study, each exposure will consists of 5-minute tape-recorded scripts recounting the recent stressful (or neutral) events in the participants' lives, including cognitions and physical feelings. Stress and neutral conditions will be randomized, counterbalanced and a two-hour interval between conditions will avoid carryover effects.

The following measures of mood, urge to drink, and physiological reactivity will be administered at baseline and post-imagery for both the stress and neutral conditions: (1) The Profile of Mood States, Short Version (POMS) will collect data on (a) negative mood; and (b) tension during the stress manipulation; (2) The Alcohol Urge Questionnaire (AUQ) will measure subjective levels of alcohol cravings at the present moment (i.e., following stress imagery); (3) Cortisol levels will be assessed by collecting saliva samples and heart rate and blood pressure will be measured using a Dynamap Adult Vital Signs Monitor. Therapeutic efficacy of ibudilast will be gauged based on the compound's ability to attenuate or abolish stress induced cravings for alcohol consumption.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of treating alcoholism or a symptom thereof in a subject diagnosed with alcoholism, the method comprising administering to the subject a therapeutically effective amount of ibudilast or a pharmaceutically acceptable salt thereof.

2. The method of claim 1, wherein the ibudilast or the pharmaceutically acceptable salt thereof is administered orally.

3. The method of claim 1, wherein the ibudilast or the pharmaceutically acceptable salt thereof is administered at a therapeutically effective daily dose from about 3 mg to about 120 mg of ibudilast or a free base equivalent amount thereof.

4. The method of claim 3, wherein the therapeutically effective daily dose is 60 mg of ibudilast or a free base equivalent amount thereof.

5. The method of claim 3, wherein the therapeutically effective daily dose is 100 mg of ibudilast or a free base equivalent amount thereof.

6. The method of claim 3, wherein the therapeutically effective daily dose is administered as a single dose or is divided into two, three, or four doses.

7. The method of claim 6, wherein the therapeutically effective daily dose is 100 mg or less of ibudilast or a free base equivalent amount thereof and is divided into two doses.

8. The method of claim 1, wherein the ibudilast is administered as part of a combination therapy that includes one or more other therapeutic agents.

9. The method of claim 8, wherein the one or more other therapeutic agents is selected from the group consisting of acamprosate, naltrexone, quetiapine, disulfiram, ondansetron, varenicline, topiramate, prazocin, sertraline and levatiracetam.

10. The method of claim 1, wherein the subject is human.

11. A method of maintaining abstinence from alcohol consumption in a subject in need of, the method comprising administering to the subject a therapeutically effective amount of ibudilast or a pharmaceutically acceptable salt thereof.

12. The method of claim 11 in which the ibudilast or a pharmaceutically acceptable salt thereof is administered once daily, twice daily, thrice daily or four times daily.

13. The method of claim 11 in which the subject has been alcohol-free for 5 days or more, 10 days or more, 20 days or more, or 30 days or more prior to receiving ibudilast.

14. The method of claim 13 in which the subject remains alcohol-free for at least another 30 days, at least another 60 days, or at least another 120 days after receiving ibudilast.

15. The method of claim 11, wherein the administration of ibudilast reduces the number of alcoholic drinks consumed per day from greater than 5 drinks/day to less than 4 drinks/day, less than 3 drinks/day, less than 2 drinks/day, or less than 1 drink/day.

16. The method of claim 15, wherein the administration of ibudilast completely eliminates the urge to consume alcohol.

17. The method of claim 11 in which the amount of the subject's daily intake of water remains substantially unaltered.

18. The method of claim 11 in which the ibudilast or the pharmaceutically acceptable salt thereof is formulated as a delayed-release tablet or capsule.

19. The method of claim 18 in which the tablet or capsule contains from 10 mg to 50 mg of ibudilast or a free base equivalent amount thereof as an active ingredient.

20. The method of claim 18 in which a plasma half-life of ibudilast is observed to be about 19 hours.

21. A method of treating a subject suffering from withdrawal from alcohol, the method comprising administering to the subject a therapeutically effective amount of ibudilast or a pharmaceutically acceptable salt thereof, wherein said administration lowers or eliminates withdrawal associated behavioral changes selected from the group consisting of feelings of nervousness, hyperexcitability, sleep disturbances and dysphoric mood.

22. The method of claim 21, wherein said administration lowers or eliminates withdrawal associated behavioral changes for a time period from at least 5 days to at least 180 days.

23. A method of treating a subject suffering from withdrawal from alcohol or a symptom thereof, the method comprising administering to the subject a therapeutically effective amount of ibudilast or a pharmaceutically acceptable salt thereof.

24. The method of claim 23, wherein the ibudilast or the pharmaceutically acceptable salt thereof is administered orally.

25. The method of claim 23, wherein the ibudilast or the pharmaceutically acceptable salt thereof is administered at a therapeutically effective daily dose from about 3 mg to about 120 mg of ibudilast or the free base equivalent amount thereof.

26. The method of claim 25, wherein the therapeutically effective daily dose is 60 mg of ibudilast or the free base equivalent amount thereof.

27. The method of claim 25, wherein the therapeutically effective daily dose is 100 mg of ibudilast or the free base equivalent amount thereof.

28. The method of claim 25, wherein the therapeutically effective daily dose is administered as a single dose or is divided into two, three, or four doses.

29. The method of claim 28, wherein the therapeutically effective daily dose is 100 mg or less of ibudilast or the free base equivalent amount thereof and is divided into two doses.

30. The method of claim 23, wherein ibudilast is administered as part of a combination therapy that includes one or more other therapeutic agents.

31. The method of claim 30, wherein the one or more other therapeutic agents is selected from the group consisting of acamprosate, naltrexone, quetiapine, disulfiram, ondansetron, varenicline, topiramate, prazocin, sertraline and levatiracetam.

32. A method of reducing alcohol consumption in a subject suffering from alcoholism, the method comprising administering to the subject a therapeutically effective amount of ibudilast or a pharmaceutically acceptable salt thereof.

33. The method of claim 32, wherein the ibudilast is or the pharmaceutically acceptable salt thereof administered orally.

34. The method of claim 32, wherein the ibudilast or the pharmaceutically acceptable salt thereof is administered at a therapeutically effective daily dose from about 3 mg to about 120 mg of ibudilast or the free base equivalent amount thereof.

35. The method of claim 34, wherein the therapeutically effective daily dose is 60 mg of ibudilast or the free base equivalent amount thereof.

36. The method of claim 34, wherein the therapeutically effective daily dose is 100 mg of ibudilast or the free base equivalent amount thereof.

37. The method of claim 34, wherein the therapeutically effective daily dose is administered as a single dose or is divided into two, three, or four doses.

38. The method of claim 37, wherein the therapeutically effective daily dose is 100 mg or less of ibudilast or the free base equivalent amount thereof and is divided into two doses.

39. The method of claim 32, wherein the ibudilast or the pharmaceutically acceptable salt thereof is administered as part of a combination therapy that includes one or more other therapeutic agents.

40. The method of claim 39, wherein the one or more other therapeutic agents is selected from the group consisting of acamprosate, naltrexone, quetiapine, disulfiram, ondansetron, varenicline, topiramate, prazocin, sertraline and levatiracetam.
